(19) United States
(12) Patent Application Publication
(10) Pub. No.: US 2011/0152840 A1
(43) Pub. Date: Jun. 23, 2011

(54) METHODS FOR REDUCING THE OCCURRENCE OF PRETERM DELIVERY AND OTHER PREGNANCY-RELATED CONDITIONS

(75) Inventors: Chang Lee, Maryland Heights, MO (US); Robert Birch, St. Charles, MO (US)

(73) Assignee: DRUGTECH CORPORATION, Wilmington, DE (US)

(21) Appl. No.: 12/971,900

(22) Filed: Dec. 17, 2010

Related U.S. Application Data
(60) Provisional application No. 61/289,572, filed on Dec. 23, 2009.

(51) Int. Cl. A61M 37/00

(52) U.S. Cl. 604/522

(57) ABSTRACT

The present invention relates to methods and kits for reducing the occurrence of preterm delivery and other pregnancy-related conditions in pregnant female subjects exhibiting one or more risk factors for preterm delivery and other pregnancy-related conditions. For example, the present invention relates to methods for reducing the occurrence of preterm delivery in a pregnant female subject having no history of preterm delivery and exhibiting one or more risk factors for preterm delivery (e.g., smoking during pregnancy). The methods and kits provide for the administration of a steroid hormone to the pregnant female subject.

### CROSS-REFERENCE TO RELATED APPLICATIONS

This application claims the benefit of U.S. Provisional Application No. 61/289,572, filed Dec. 23, 2009, the entirety of which is herein incorporated by reference.

### FIELD OF THE INVENTION

The present invention relates to methods and kits for reducing the occurrence of preterm delivery and other pregnancy-related conditions in pregnant female subjects exhibiting one or more risk factors for preterm delivery and other pregnancy-related conditions. For example, the present invention relates to methods for reducing the occurrence of preterm delivery in a pregnant female subject having no history of preterm delivery and exhibiting one or more risk factors for preterm delivery (e.g., smoking during pregnancy). The methods and kits of the present invention provide for the administration of a steroid hormone to the pregnant female subject.

### BACKGROUND OF THE INVENTION

Preterm delivery is a major health problem in the United States and worldwide. Preterm delivery is often defined as delivery before 37 completed weeks of gestation and has been reported to be the major determinant of infant mortality in developed countries. Preterm delivery is more common in the United States than in many other developed countries, and is predominantly responsible for the relatively high rate of infant mortality in the United States as compared to many other developed countries. Over the past two decades, the rate of preterm delivery in the United States has been reported to have increased from 9% to 12%. In addition to preterm delivery, various other pregnancy-related conditions are major health problems in the United States and worldwide. These include, for example, the delivery of low birth weight neonates, delivery of small for gestational age neonates, pregnancy-related complications, fetal mortality, neonatal morbidity, neonatal mortality, infant morbidity, infant mortality, and childhood developmental delays. As used herein, the terms “neonates”, “neonatal,” etc. refers to the time period within the first 4 weeks of a live birth.

Preterm delivery and other pregnancy-related conditions such as the delivery of low birth weight neonates and/or small for gestational age neonates have serious health, societal, and economic costs. For example, preterm delivery and the delivery of low birth weight neonates and/or small for gestational age neonates can lead to neonatal morbidity, longer stays in the neonatal intensive care unit, and a higher risk of long term morbidities including, for example, cerebral palsy, mental retardation, and learning disabilities.

A number of risk factors for preterm delivery and other pregnancy-related conditions (e.g., delivery of low birth weight and/or small for gestational age neonates) have been identified. For example, women who have had a previous spontaneous preterm delivery are at high risk for preterm delivery in subsequent pregnancies. Other risk factors for preterm delivery include: tobacco use during pregnancy (e.g., smoking); infection; multiple gestations (twins, triplets, etc.); alcohol use, abuse, or dependence during pregnancy; substance use, abuse, or dependence during pregnancy; poor nutrition during pregnancy; stress, anxiety, and/or depression; insufficient weight gain during pregnancy; advanced maternal age; and low socio-economic status. Tobacco use or exposure, in particular smoking, during pregnancy is a significant risk factor for preterm delivery and other undesirable maternal, fetal, and neonatal outcomes. In addition to preterm delivery, smoking during pregnancy is associated with, for example, delivery of low birth weight neonates, delivery of small for gestational age neonates, pregnancy-related complications (e.g., placental abruption), stillbirth, increased risk of spontaneous abortion, and ectopic pregnancies. Smoking during pregnancy may also result in an increased risk of sudden infant death syndrome (SIDS) and an increased risk of behavioral disorders during childhood.

Other than smoking cessation, no intervention is known to ameliorate the maternal, fetal, and neonatal effects of smoking during pregnancy. According to the most recent Centers for Disease Control (CDC) estimates, as of 2005, an estimated 13.8% of women (i.e., more than 1 in 8) in the United States smoked during pregnancy. These data are based on self-reporting and, therefore, are thought to be underestimates of the rate of smoking among pregnant women. Smoking intervention strategies during pregnancy have had limited success. Therefore, significant risks associated with the maternal, fetal, and neonatal effects of smoking during pregnancy persist. Thus, there exists a need in the art for methods for reducing the occurrence of preterm delivery associated with smoking during pregnancy. There also exists a need in the art for methods for reducing the risk of other pregnancy related conditions (e.g., delivery of low birth weight and/or small for gestational age neonates) associated with smoking during pregnancy.

Furthermore, even women who do not themselves smoke may be at risk for preterm delivery and other undesirable outcomes due to tobacco exposure via second-hand smoke (i.e., passive smoking or environmental tobacco smoke (ETS)) or third-hand smoke (e.g., tobacco smoke residue on environmental surfaces). Thus, women who live with persons who smoke or work in an environment in which they are exposed to tobacco smoke may be particularly at risk for preterm delivery and other undesirable maternal, fetal, and/or neonatal outcomes.

Smoking during pregnancy often correlates with other behaviors and risk factors believed to contribute to preterm delivery and other pregnancy-related conditions. These include the various risk factors noted above such as, for example, alcohol use, abuse, or dependence during pregnancy; substance use, abuse, or dependence during pregnancy; poor nutrition during pregnancy; stress, anxiety, and/or depression; insufficient weight gain during pregnancy; advanced maternal age; and low socio-economic status. For example, as compared to non-smokers, it has been reported that women who smoked during pregnancy were less likely to have completed 12 years of school, less likely to have paid full-time or part-time employment, had lower body mass indices, and were more likely to suffer from depression, anxiety or perceived stress. In addition the women who smoked throughout pregnancy were more likely to use alcohol (8% vs. 4%) and less likely to take folic acid (65% vs. 75%) or use multivitamins (41% vs. 59%). Thus, there exists a need in the art for methods for reducing the occurrence of preterm delivery and other pregnancy-related conditions (e.g., delivery of low birth weight and/or small for gestational age neonates) associated with such risk factors.

### SUMMARY OF THE INVENTION

Briefly, therefore, the present invention is directed to methods for reducing the occurrence of preterm delivery and/or other pregnancy-related conditions. Generally, the methods comprise administering a pharmaceutical composition comprising a steroid hormone to a pregnant female subject exhibiting one or more risk factors for preterm delivery and/or other pregnancy-related conditions. The risk factors are generally selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

Various embodiments of the present invention are directed to methods for reducing the occurrence of preterm delivery.

In one embodiment, the method comprises administering a pharmaceutical composition comprising a steroid hormone to a pregnant female subject having no history of preterm delivery and exhibiting one or more risk factors for preterm delivery. The one or more risk factors are selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

In another embodiment, the method comprises administering a pharmaceutical composition comprising a steroid hormone to a pregnant female subject having no history of preterm delivery, wherein the pregnant female subject is at risk for preterm delivery due to exposure to tobacco smoke.

Various embodiments of the present invention are directed to methods for reducing the occurrence of one or more conditions selected from the group consisting of delivery of low birth weight neonates, delivery of small for gestational age neonates, pregnancy-related complications, fetal mortality, neonatal morbidity, neonatal mortality, infant morbidity, infant mortality, childhood developmental delays, and combinations thereof. The method comprises administering a pharmaceutical composition comprising a steroid hormone to a pregnant female subject having no history of preterm delivery and exhibiting one or more risk factors for the one or more conditions. The one or more risk factors are selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

The present invention is also directed to kits comprising (i) a pharmaceutical composition comprising a steroid hormone and one or more pharmaceutically acceptable excipients; and (ii) instructions for administering the pharmaceutical composition to a pregnant female subject to reduce the occurrence of preterm delivery. The pregnant female subject has no history of preterm delivery and exhibits one or more risk factors for preterm delivery selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

In another embodiment, the kit comprises (i) a pharmaceutical composition comprising a steroid hormone and one or more pharmaceutically acceptable excipients; and (ii) instructions for administering the pharmaceutical composition to a pregnant female subject to reduce the occurrence of preterm delivery. The pregnant female subject has no history of preterm delivery, and is at risk for preterm delivery due to exposure to tobacco smoke.

Various embodiments of the present invention are directed to kits comprising (i) a pharmaceutical composition comprising a steroid hormone and one or more pharmaceutically acceptable excipients; and (ii) instructions for administering the pharmaceutical composition to a pregnant female subject to reduce the occurrence of one or more conditions selected from the group consisting of delivery of low birth weight neonates, delivery of small for gestational age neonates, pregnancy-related complications, fetal mortality, neonatal morbidity, neonatal mortality, infant morbidity, infant mortality, childhood developmental delays, and combinations thereof. The pregnant female subject has no history of preterm delivery and exhibits one or more risk factors for the one or more conditions. The one or more risk factors are selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

The present invention is further directed to methods for treating a smoking-related disease or disorder. In one embodiment, the method comprises administering a pharmaceutical composition comprising a progestogen to a subject suffering from a smoking-related disease or disorder.

### DETAILED DESCRIPTION OF THE PREFERRED EMBODIMENTS

Described herein are methods, compositions, and kits suitable for reducing the occurrence of preterm delivery and various other pregnancy-related conditions such as delivery of low birth weight neonates, delivery of small for gestational age neonates, pregnancy-related complications, fetal mortality, neonatal morbidity, neonatal mortality, infant morbidity, infant mortality, and childhood developmental delays. More specifically, the methods, compositions, and kits of the present invention are currently believed to be effective for reducing the occurrence of preterm delivery and/or one or more other pregnancy-related conditions in a pregnant female subject at risk for preterm delivery and/or one or more pregnancy-related conditions based on exhibiting one or more risk factors. Risk factors for preterm delivery and various other pregnancy-related conditions include exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof. A primary risk factor is smoking by the pregnant female subject during pregnancy.

Despite the serious consequences of preterm delivery, there has been little progress in the treatment of women who are at increased risk for preterm delivery. Trials of reduced activity, tocolytic therapy (i.e., therapy to reduce or stop uterine contractions), antibiotic therapy, and other treatments have generally failed to demonstrate an effective and reproducible method for reducing the occurrence (i.e., preventing) preterm delivery. However, prophylactic treatment with progestational compounds including, for example, 17alpha-hydroxyprogesterone caproate (17-HPC), has shown promise in certain subpopulations of pregnant women. For example, in Meis et al., New England J. Med., Prevention of Recurrent Preterm Delivery by 17 Alpha-Hydroxyprogesterone Caproate, 348(24):2379-2385 (Jun. 12, 2003) (incorporated by reference herein for all relevant purposes), treatment with 17-HPC was found to reduce the risk of preterm delivery in women having a documented history of spontaneous preterm delivery, and who are therefore at a greatly increased risk for preterm delivery in subsequent pregnancies. However, most preterm deliveries occur in women with no previous preterm delivery. Advantageously in accordance with the present invention, the methods detailed herein are believed to significantly reduce the occurrence of preterm delivery in subjects having no history of preterm delivery. This treatment is specifically adapted to reducing the occurrence of preterm delivery in pregnant female subjects exhibiting at least one risk factor for preterm delivery. Thus, methods of the present invention are suitable for reducing the impact of these risk factors on causing preterm delivery. Preferably, the methods of the present invention are suitable for substantially negating the effect of at least one risk factor associated with preterm delivery. In this manner, it is currently believed that the methods of the present invention may effectively modulate the effect of a risk factor such as smoking on the occurrence of preterm delivery. Thus, various embodiments of the present invention are directed to methods for reducing the occurrence of preterm delivery in a pregnant female subject having no history of preterm delivery and exhibiting one or more risk factors for preterm delivery. In these and various other embodiments, the pregnant female subject to be treated is experiencing her first pregnancy (i.e., the subject is a primigravida). Various other embodiments are directed to treating subjects that have already experienced one or more live births without a preterm delivery. In a preferred embodiment, the method is directed to reducing the occurrence of preterm delivery in a pregnant female subject having no history of preterm delivery, but exhibiting a risk factor for preterm delivery due to exposure to tobacco smoke (e.g., smoking during pregnancy).

As noted, 17-HPC has been tested for effectiveness in reducing preterm delivery in a study of patients exhibiting a history of preterm delivery as described, for example, in Meis et al. (2003). As is well-known in to one skilled in the art, smoking causes various physical side effects including, for example, constriction of blood vessels which may lead to various maternal side effects such as ischemia and an increased placental weight. Furthermore, it has been reported that tobacco smoke is an oxidative stress, and reduces histone deactelylase expression and activity, which is necessary for inflammatory gene transcription and adequate inhibition of cytokine production by glucocorticoids. See, e.g., Ito et al., Cigarette Smoking Reduces Histone Deacetylase 2 Expression, Enhances Cytokine Expression, and Inhibits Glucocorticoid Actions in Alveolar Macrophages, FASEB J. 15(6):1110-2 (April 2001), which is incorporated by reference herein for all relevant purposes. Glucocorticoids are important in maintaining pregnancy. Without being bound to a particular theory, it is currently believed that a skilled artisan would expect that the tobacco smoke toxicants would reduce glucocorticoid action, leading to an increased risk of preterm delivery, low birth weight or other pregnancy-related conditions. Again, without being bound to a particular theory, it is further currently believed that a skilled artisan would expect that these actions on glucocorticoid function would decrease the effectiveness of a steroid hormone treatment such as 17-HPC. However, in accordance with the present invention it has been discovered that the physical side effects of smoking such as blood vessel constriction and the molecular effects of the tobacco smoke toxicants do not inhibit the effectiveness of 17-HPC in reducing the occurrence of preterm delivery. It has further been discovered that administration of 17-HPC to a pregnant female subject known to be a smoker substantially reduces the effect of this risk factor on causing preterm deliveries, and it is believed that 17-HPC has a similar effect in pregnant female subjects who may not themselves smoke, but who are at risk for exposure to tobacco smoke. In particular, administering 17-HPC to pregnant women who are smokers has been shown to significantly reduce the risk of preterm delivery, as measured by the proportion of neonates born at less than 37 weeks of gestational age. Thus, in accordance with the present invention, it is currently believed that administration of 17-HPC can significantly reduce the effect of risk factors such as smoking on preterm deliveries, regardless of any history of preterm delivery associated with the subject. Since most preterm deliveries occur in subjects that do not have a history of preterm delivery, the present discovery represents a significant advance in the art.

The methods of the present invention are also directed to reducing the occurrence of one or more pregnancy-related conditions in a pregnant female subject exhibiting one or more risk factors for at least one of the pregnancy related conditions. These pregnancy-related conditions include delivery of low birth weight neonates, delivery of small for gestational age neonates, pregnancy-related complications, fetal mortality, neonatal morbidity, neonatal mortality, infant morbidity, infant mortality, and childhood developmental delays. As noted, the one or more risk factors for these conditions include exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof. In various embodiments, the pregnant female subject does not have a history of any of the pregnancy-related conditions. In still other embodiments, the pregnant female subject has a history of one or more of the conditions.

### I. Pregnancy-Related Conditions

A. Preterm Delivery

As noted, preterm delivery is a major health problem in the U.S. and worldwide. Preterm delivery is often defined to include any delivery before 37 weeks or before 35 weeks of gestation. The gestational age of an embryo or fetus is generally calculated from the date of the woman's last menstrual period or from 14 days before conception if the date of conception is known. For purposes of determining the effectiveness of the methods of the present invention, preterm delivery can be defined as any live birth occurring prior to 37 weeks of gestation, prior to 36 weeks of gestation, or prior to 35 weeks of gestation. Since viability may occur for live births prior to 35 weeks of gestation, preterm delivery may also be defined as any live birth occurring between 20 and 36 weeks of gestation.

B. Low Birth Weight/Small for Gestational Age Neonates

Neonates having a relatively low birth weight and/or relatively small size are generally associated with a higher risk of various complications as compared to neonates having a weight and/or size within normal ranges, including an increased risk for neonatal morbidity and mortality, and infant morbidity and mortality. As used herein, the term “low birth weight neonates” encompasses low birth weight neonates (neonates having a weight at birth of less than about 2500 g (about 5.5 pounds)), very low birth weight neonates (neonates having a weight at birth of less than about 1500 g (about 3.3 pounds)), and extremely low birth weight neonates (neonates having a weight at birth of less than about 1000 g (about 2.2 pounds)). A neonate is suitably classified as a small for gestational age neonate if his or her weight at birth is below the 10th percentile for gestational age, as measured according to the accepted standards published by Battaglia et al., or if birth weight and/or length are at least 2 standard deviations (SDs) below the mean for gestational age, as described by Lee et al. See Battaglia et al., A Practical Classification of Newborn Infants by Weight and Gestational Age, J. Pediatrics 71(2):159-63 (August 1967) and Lee et al., International Small for Gestational Age Advisory Board Consensus Development Conference Statement: Management of Short Children Born Small for Gestational Age, Apr. 24-Oct. 1, 2001, Pediatrics 111(6 Pt. 1):1253-61 (June 2003), both of which are incorporated by reference herein for all relevant purposes.

C. Pregnancy-Related Complications

In addition to, or separately from reducing the occurrence of preterm delivery, delivery of low birth weight neonates, etc., the methods of the present are currently believed to be effective for reducing the occurrence of one or more pregnancy-related complications. Pregnancy-related complications that can be prevented by use of the methods and kits of the present invention include, for example, placental abruption, placenta previa, and hypertension-related disorders (e.g., preeclampsia and eclampsia). These complications are generally known to contribute to preterm delivery, delivery of low birth weight neonates, etc. Thus, reducing the occurrence of these complications likewise reduces the occurrence of preterm delivery, delivery of low birth weight neonates, etc.

D. Mortality

Fetal mortality includes any death of a fetus at 20 weeks of gestation or later or any death of a fetus weighing more than 500 g. Fetal mortality includes both antepartum deaths (i.e., deaths occurring before birth) and intrapartum deaths (i.e., deaths occurring during labor and delivery).

Neonatal mortality refers to the death of a live-born neonate within the first 28 days of life. Neonatal mortality includes both early neonatal mortality (i.e., death of a live-born neonate within the first seven days of life) and late neonatal mortality (i.e., death of a live-born neonate after the first seven days of life but within the first 28 days of life). Together, fetal mortality and early neonatal mortality are often referred to as “perinatal mortality.” Thus, “perinatal mortality” refers to deaths occurring between 20 weeks of gestation and the end of the 7th day after delivery.

Infant mortality includes deaths which occur after 28 days of life, but before one year.

E. Morbidity and Developmental Delays

Neonatal morbidity and infant morbidity refer to any disease, disorder, symptom, or other undesirable outcome occurring in a neonate or an infant, respectively. Developmental delays occur when children have not yet reached expected developmental milestones by the expected time period. Neonatal morbidity, infant morbidity, and childhood developmental delays encompass a number of conditions affecting neonates, infants, and/or children, including, but not limited to transient tachypnea, respiratory distress syndrome, bronchopulmonary dysplasia, a need for ventilatory support/mechanical ventilation, a need for supplemental oxygen, intraventricular hemorrhage, necrotizing enterocolitis, patent ductus arteriosus, retinopathy, sepsis, sudden infant death syndrome (SIDS), cerebral palsy, mental retardation, learning disabilities, and behavioral disorders. Various additional diagnoses associated with neonatal morbidity, infant morbidity, and/or childhood developmental delays include anemia, arthritis, asthma, diabetes, diarrhea, colitis, ear infections, eczema, food or digestive allergies, hay fever, respiratory allergies, seizures, severe headaches or migraines, sickle cell disease, and stuttering and stammering. Other conditions include communication problems, problems with problem solving, attention or learning problems (e.g., attention-deficit hyperactivity disorder (ADHD)), autism, problems carrying out activities and problems with coordination.

### II. Risk Factors

A variety of risk factors associated with the above-listed pregnancy-related conditions are detailed below. Various risk factors listed below are in connection with exposure to tobacco (e.g., tobacco smoke or tobacco smoke residue). Other risk factors that may contribute to and/or cause one or more pregnancy-related conditions include substance use or abuse or dependence, alcohol use or abuse or dependence, stress, poor nutritional status, insufficient weight gain during pregnancy, advanced maternal age, low socio-economic status, and combinations thereof. Behaviors unfavorable to a subject's health such as smoking tend to cluster (e.g., women who smoke are also more likely to have poor diets). Thus, many women exhibit more than one risk factor for the pregnancy-related conditions, which may increase the risk of occurrence of the pregnancy-related conditions. For example, the occurrence of more than one of the following risk factors are commonly exhibited by a single subject: exposure to tobacco smoke, stress, poor nutritional status, low socio-economic status, alcohol use, abuse, or dependence. Thus, in various preferred embodiments the methods of the present invention are directed to reducing the occurrence of one or more pregnancy-related conditions in a pregnant female subject exhibiting at least one risk factor selected from the group consisting of exposure to tobacco smoke, stress, poor nutritional status, low socio-economic status, alcohol use or abuse or dependence, and combinations thereof.

A. Tobacco

One significant risk factor for preterm delivery and the other pregnancy-related conditions is exposure of the pregnant female to tobacco smoke during pregnancy. This exposure may occur in many forms. Exposure to tobacco smoke includes smoking of tobacco products by the pregnant female subject herself, as well as passive smoking via the inhalation of smoke from tobacco products used by others (commonly referred to as second-hand smoke or environmental tobacco smoke). In either case, the tobacco smoke may be smoke generated by the use of, for example, a cigarette, a cigar, or a pipe, or any other implement which generates smoke from tobacco. A primary means of exposure of subjects to tobacco smoke in accordance with the present invention is smoking by the pregnant female subject.

Tobacco smoke residue typically contains nicotine, heavy metals, carcinogens, carbon monoxide, reactive oxygen species, and other toxicants which can contribute to or cause one or more pregnancy-related complications. Exposure to tobacco smoke residue or the use of smokeless tobacco may pose similar risks to the pregnant female subject as exposure to tobacco smoke. Exposure to tobacco smoke residue includes exposure to toxicants which accumulate on environmental surfaces in areas wherein tobacco products have been smoked. Tobacco smoke residue is commonly referred to as “third-hand smoke.” Tobacco smoke residue can accumulate on virtually any environmental surface, including, but not limited to, hair, clothing, furniture, carpeting, and automobile upholstery.

Use of smokeless tobacco is also a risk factor for the above-noted pregnancy-related conditions and includes use of any type of tobacco that is consumed other than by smoking. For instance, smokeless tobacco includes, but is not limited to, dipping tobacco, chewing tobacco, snuff, creamy snuff, snus, tobacco gum, dissolvable tobacco, topical tobacco paste, and tobacco water.

B. Substance Use, Abuse, and/or Dependence

Substance use, abuse, or dependence includes the use or abuse of, or the dependence on, drugs commonly referred to as “street drugs” (e.g., marijuana and cocaine) and/or the use or abuse of, or the dependence on, prescription drugs other than as directed by a physician. Substance use refers to use which is sufficient to result in a positive result on any test commonly used for screening for substance use including, for example, blood tests, urine tests, etc. Substance abuse and substance dependence are suitably diagnosed according to the diagnostic criteria well known to those skilled in the art, such as those set forth in the Diagnostic and Statistical Manual of Mental Disorders (American Psychiatric Association, 4th ed., text revision) (DSM-IV-TR), which is incorporated by reference herein for all relevant purposes. Substance use, abuse, or dependence may suitably refer to the use or abuse of, or the dependence on, for example, opioids, depressants, hallucinogens, stimulants, hypnotics, analgesics, inhalants, sedatives, anxiolytics, or combinations thereof. For instance, substance use, abuse, or dependence may include, but is not limited to, the use or abuse of, or the dependence on, marijuana, cocaine (in powder or crack cocaine form), heroin, methamphetamine, anabolic steroids, 3,4-methylenedioxymethamphetamine (MDMA (ecstasy)), psilocybin, psilocin, lysergic acid diethylamide (LSD), morphine, oxycodone, phencyclidine (also referred to as phenylcyclohexylpiperidine or PCP.

C. Alcohol Use, Abuse, and/or Dependence

Alcohol use, abuse, or dependence generally includes the use or abuse of, or the dependence on, any alcohol-containing product, such as beer, wine, or liquor. Alcohol use may specifically refer to confirmed use of alcohol during pregnancy. High risk alcohol use during pregnancy is defined as confirmed use of alcohol sufficient to produce high blood alcohol levels (100 mg/dL or greater) delivered at least weekly in early pregnancy. Alcohol abuse and alcohol dependency are suitably diagnosed according to the diagnostic criteria well known to those skilled in the art, such as those set forth in the Diagnostic and Statistical Manual of Mental Disorders, which is incorporated by reference herein for all relevant purposes.

D. Stress/Anxiety/Depression

Experiencing relatively high stress levels may put pregnant women at an increased risk for one or more of the above-noted pregnancy-related conditions. Stress levels are suitably measured by a method well known to one skilled in the art, for example, by psychometric scales including the stress component of the Abbreviated Scale for the Assessment of Psychosocial Status in Pregnancy tool, the Stressful Life Events scale (part of the CDC's Pregnancy Risk Assessment and Monitoring System (PRAMS)) and the Modified Life Experiences Survey. A stress level exceeding the pre-defined values for one of these scales would generally be considered to increase the risk for the pregnancy-related conditions discussed above. Stress may be caused, for example, by life events such as divorce, illness, injury, job loss, or the like.

In addition, women who have been diagnosed with anxiety and/or depression according to the standards generally used by medical professionals (e.g., those set forth in the Diagnostic and Statistical Manual of Mental Disorders, which is incorporated by reference herein for all relevant purposes) may also be at increased risk for the pregnancy-related conditions discussed above.

E. Nutritional Status

Poor nutritional status may put a pregnant female at an increased for the one or more of the above-noted pregnancy-related conditions. Nutritional status may be assessed by weight gain during pregnancy based on pre-pregnancy body mass index (BMI) according to the Institute of Medicine recommendations. See Institute of Medicine, Weight Gain During Pregnancy: Reexamining the Guidelines (2009), which is incorporated by reference herein for all relevant purposes. For example, a pregnant female subject will generally be considered to have a poor nutritional status if weight gain during pregnancy is insufficient according to these guidelines.

F. Weight Gain

A pregnant female subject is considered to have gained insufficient weight during pregnancy if the subject had a pre-pregnancy BMI of less than about 18.5 kg/m² and total weight gain was less than about 12.7 kg (i.e., less than about 28 lbs), had a pre-pregnancy BMI of from about 18.5 to about 24.9 kg/m² and total weight gain was less than about 11.3 kg (i.e. less than about 25 lbs), had a pre-pregnancy BMI of about 25.0 to about 29.9 kg/m² and total weight gain was less than about 6.8 kg (i.e., less than about 15 lbs), or had a pre-pregnancy BMI of at least about 30.0 kg/m² and total weight gain was less than about 5.0 kg (i.e., less than about 11 lbs). Additionally or alternatively, a pregnant female subject in the second or third trimester is considered to have gained insufficient weight during pregnancy if the subject had a pre-pregnancy BMI of less than about 18.5 kg/m² and total weight gain during the second and third trimesters was less than about 0.45 kg/week (i.e., less than about 1 lb/week), had a pre-pregnancy BMI of from about 18.5 to about 24.9 kg/m² and total weight gain during the second and third trimesters was less than about 0.36 kg/week (i.e., less than about 0.8 lbs/week), had a pre-pregnancy BMI of from about 25.0 to about 29.9 kg/m² and total weight gain during the second and third trimesters was less than about 0.23 kg/week (i.e., less than about 0.5 lbs/week), or had a pre-pregnancy BMI of at least about 30.0 kg/m² and total weight gain during the second and third trimesters was less than about 0.18 kg/week (i.e., less than about 0.4 lbs/week).

G. Maternal Age

Generally, as maternal age increases so too does the risk of occurrence of preterm delivery and/or one or more other pregnancy-related conditions. By advanced maternal age, it is meant that the pregnant female subject is at least 35 years of age at the time of delivery.

H. Socio-Economic Status

A pregnant female subject is suitably considered to have a low socioeconomic status if the pregnant female subject's family and/or household income is at or below the federal poverty level or if the pregnant female subject is eligible for the Medicaid program.

### III. Steroid Hormones

Generally in accordance with the present invention, the method for reducing the occurrence of preterm delivery and/or one or more other pregnancy-related conditions comprises administering a pharmaceutical composition comprising a steroid hormone. Typically, the steroid hormone is a progestogen. The progestogen may be a naturally occurring progestogen or a synthetic progestogen (i.e., a progestin). For example, the progestogen is suitably a pregnane or a derivative thereof, a norpregnane or a derivative thereof, an estrane or a derivative thereof, a gonane or a derivative thereof, or a combination thereof. Generally, the progestogen for use in accordance with the present invention is selected from the group consisting of progesterones; retroprogesterones; 17alpha-hydroxyprogesterone derivatives (pregnanes); 17alpha-hydroxynorprogesterone derivatives and 19-norprogesterone derivatives (norpregnanes); 19-nortestosterone derivatives (estranes and gonanes); and combinations thereof. Specific examples of progestogens that may be used in the methods and kits of the present invention include, but are not limited to, 17alpha-hydroxyprogesterone or a derivative thereof, natural progesterone, dydrogesterone or a derivative thereof, medrogestone or a derivative thereof, medroxyprogesterone or a derivative thereof, megestrol or a derivative thereof, chlormadinone or a derivative thereof, cyproterone or a derivative thereof, gestonorone or a derivative thereof, nomegestrol or a derivative thereof, demegestone or a derivative thereof, promegestone or a derivative thereof, nestorone or a derivative thereof, trimegestone or a derivative thereof, norethisterone or a derivative thereof, lynestrenol or a derivative thereof, ethynodiol or a derivative thereof, norgestrel or a derivative thereof, levonorgestrel or a derivative thereof, desogestrel or a derivative thereof, etonogestrel (3-keto-desogestrel) or a derivative thereof, gestodene or a derivative thereof, norgestimate or a derivative thereof, norelgestromin (17-deacetyl norgestimate) or a derivative thereof, dienogest or a derivative thereof, drospirenone or a derivative thereof, norethindrone or a derivative thereof, norethynodrel or a derivative thereof, and combinations thereof.

Derivatives of medroxyprogesterone, megestrol, chlormadinone, cyproterone, gestonorone, nomegestrol, norethisterone, and ethynodiol include carboxylic acid esters of these compounds. Suitable esters of these compounds include, for example, medroxyprogesterone acetate, megestrol acetate, chlormadinone acetate, cyproterone acetate, gestonorone caproate, nomegestrol acetate, norethisterone acetate, and ethynodiol diacetate.

One suitable progestin for use in the methods and kits of the present invention is 17alpha-hydroxyprogesterone or a pharmacologically active derivative thereof. Suitable derivatives of 17alpha-hydroxyprogesterone include esters of 17alpha-hydroxyprogesterone, and in particular 17alpha-hydroxyprogesterone caproate (17-HPC), which has been approved for human use by the United States Food and Drug Administration (FDA). Other esters of 17alpha-hydroxyprogesterone may also suitably be used. In accordance with various preferred embodiments, the pharmaceutical composition comprises 17-HPC.

In various other embodiments, the steroid hormone may be an androgen (e.g., dehydroepiandrosterone (DHEA)), an estrogen (e.g., estradiol), a glucocorticoid (e.g., cortisol, dexamethasone, prednisone, prednisolone, methylprednisolone, betamethasone, triamcinolone, beclometasone, or fluticasone), or a mineralocorticoid, (e.g., fludrocortisone, aldosterone, or deoxycorticosterone).

### IV. Administration

Treatment of a pregnant female subject with a steroid hormone typically begins during the first or second trimester of pregnancy (i.e., during weeks 1-27 of gestation) and continues until relatively late in the third trimester or until delivery, whichever occurs first. However, it is anticipated that the benefits of the invention will still be realized even if steroid hormone treatment is not initiated until the third trimester. Thus, for example, treatment with a steroid hormone is typically initiated at between 1 week and about 35 weeks of gestation and continues until about 37 weeks of gestation, or delivery, whichever occurs first. Alternatively, the steroid hormone treatment is suitably initiated at between about 12 weeks and about 30 weeks of gestation and continues until about 36 weeks of gestation, or delivery, whichever occurs first. In some embodiments, the steroid hormone treatment is initiated at between about 16 and about 20 weeks of gestation, and continues until about 36 weeks after gestation, or delivery, whichever occurs first. In accordance with various preferred embodiments, treatment with a steroid hormone is initiated during the second or third trimester. Thus, in accordance with various preferred embodiments, treatment with a steroid hormone is initiated at 13 weeks of gestation or later (e.g., at or around 28 weeks of gestation or later).

The pharmaceutical compositions may be administered enterally or parenterally. For example, the pharmaceutical composition may be administered by subcutaneous, intravenous intraperitoneal, or intramuscular injection; rectally; transdermally; intravaginally; or orally. When administered by subcutaneous or intramuscular injection, the steroid hormone is suitably formulated as a depot formulation to allow for sustained release of the steroid hormone over an extended period of time. When a transdermal route of administration is used, delivery may suitably be, for example, via a patch, cream, gel, or spray. Intravaginal delivery suitably includes, for example, delivery via a suppository, gel, or a cream.

With respect to the frequency of administration, any frequency which achieves the desired result (i.e., preventing preterm delivery or preventing or treating another pregnancy-related condition) may be used. The frequency of administration will be determined, at least in part, by the steroid hormone(s) and/or dosage form selected. In various embodiments, the pharmaceutical composition is administered at an interval exceeding once per week. For example, the pharmaceutical composition may be administered once every other week, once monthly, once every two months, or once every three months. In various other embodiments, the pharmaceutical composition is administered once weekly, or at an interval of less than one week (e.g., daily or every other day). For example, when the steroid hormone is 17alpha-hydroxyprogesterone caproate (17-HPC), administration may suitably be via once-weekly injections of a depot formulation comprising 17-HPC and a pharmaceutically acceptable oil (e.g., castor oil). Those skilled in the art will understand that the route of administration and frequency of administration for the pharmaceutical compositions used in the methods and kits of the present invention will depend on a variety of factors including, for example, the particular steroid hormone(s) used and the formulation in which it is delivered.

The skilled artisan will also appreciate that appropriate dosing of the steroid hormone will depend on the steroid hormone(s) selected, the route of administration and dosage form, the frequency of administration, and/or the pregnancy-related condition(s) to be treated. For example, when the steroid hormone is 17alpha-hydroxyprogesterone caproate (17-HPC) and the pharmaceutical composition is administered to a pregnant female subject as a depot injection at an interval exceeding once weekly, the pharmaceutical composition typically comprises about 100 milligrams (mg) to about 3000 mg of 17-HPC. When the steroid hormone is 17-HPC and the pharmaceutical composition is administered to a pregnant female subject as a once-monthly depot injection, the composition typically comprises at least about 100 mg of 17-HPC, at least about 200 mg of 17-HPC, at least about 300 mg of 17-HPC, at least about 400 mg of 17-HPC, at least about 500 mg of 17-HPC, at least about 750 mg of 17-HPC, at least about 1000 mg of 17-HPC, at least about 1500 mg of 17-HPC, at least about 2000 mg of 17-HPC, or at least about 2500 mg of 17-HPC. In accordance with these and various other embodiments, when the steroid hormone is 17-HPC and the pharmaceutical composition is administered as a once-monthly depot injection, the composition suitably comprises less than about 3000 mg of 17-HPC, less than about 2500 mg of 17-HPC, less than about 2000 mg of 17-HPC, less than about 1500 mg of 17-HPC, less than about 1000 mg of 17-HPC, less than about 500 mg of 17-HPC, or less than about 250 mg of 17-HPC. For example, a depot formulation of 17-HPC for once-monthly injection suitably comprises from about 100 mg to about 3000 mg of 17-HPC, from about 200 mg to about 2500 mg of 17-HPC, or from about 500 mg to about 1500 mg of 17-HPC.

As another example, in embodiments where the steroid hormone is 17-HPC and the pharmaceutical composition is administered to a pregnant female subject as a once-weekly depot injection, the pharmaceutical composition suitably comprises at least about 50 mg of 17-HPC, at least about 75 mg of 17-HPC, at least about 100 mg of 17-HPC, at least about 150 mg of 17-HPC, at least about 200 mg of 17-HPC, at least about 250 mg of 17-HPC, at least about 500 mg of 17-HPC, at least about 750 mg of 17-HPC, or at least about 1000 mg of 17-HPC. In accordance with these and various other embodiments wherein the pharmaceutical composition is administered as a once-weekly depot injection, the pharmaceutical composition typically comprises less than about 1600 mg of 17-HPC, less than about 1500 mg of 17-HPC, less than about 1250 mg of 17-HPC, less than about 1000 mg of 17-HPC, less than about 800 mg of 17-HPC, less than about 500 mg of 17-HPC, or less than about 250 mg 17-HPC. For example, a depot formulation of 17-HPC for once-weekly injection suitably comprises from about 50 mg to about 1600 mg of 17-HPC, from about 100 mg to about 800 mg of 17-HPC, or about 250 mg 17-HPC.

Generally, along with the steroid hormone(s), the pharmaceutical composition contains one or more pharmaceutically acceptable excipients. For example, the pharmaceutical composition may contain one or more diluents, one or more carriers, one or more binders, one or more coatings, one or more lubricants, one or more solvents, one or more buffers, one or more preservatives, one or more flavoring agents, one or more dyes, one or more absorption enhancers, and/or one or more biodegradable polymers. The particular excipient(s) included in the pharmaceutical composition will depend on the particular steroid hormone(s) and dosage form selected, and the skilled artisan will be able to readily select appropriate excipients once the steroid hormone(s) and the dosage form therefore have been chosen. For example, 17-HPC may suitably be administered as a castor oil-based depot injection.

In addition to administration of a steroid hormone, the methods of the present invention may further comprise one or more additional treatments aimed at reducing the risk and/or occurrence of preterm delivery and/or one or more of the other pregnancy-related conditions discussed above. The one or more additional treatments typically include, for example, behavior modification, stress reduction or counseling, early access to care, administration or prescribing of one or more pharmaceutical products, administration or prescribing of one or more nutritional products, reduced physical activity, and/or one or more surgical interventions.

Behavior modification may include cognitive, emotional, and/or social/environmental interventions.

Stress reduction or counseling may include relaxation techniques, cognitive therapy, or other clinically approved techniques.

Early access to care typically includes access to prenatal care during the first trimester (≦13 weeks) of gestation.

The one or more pharmaceutical products may be selected from the group consisting of tocolytic agents, antibiotics, corticosteroids, anxiolytics, estrogen therapy, and combinations thereof.

The one or more nutritional products may be selected from the group consisting of folic acid, omega-3 fatty acids, multivitamins, iron, and combinations thereof.

Reduced physical activity refers to, for example, avoiding strenuous exercise or bed rest.

Surgical interventions may suitably include, for example, cervical cerclage.

When the pregnant female subject is at risk for preterm delivery and/or one or more other pregnancy-related conditions due to tobacco use during pregnancy (e.g., smoking), various products or therapies may be used prior to and/or concurrently with administration of one or more steroid hormones in order to bring about cessation of the tobacco use by the pregnant female subject. For example, when the tobacco use is smoking of tobacco products by the pregnant female subject herself, one or more smoking cessation products or therapies may be used prior to and/or concurrently with administration of one or more steroid hormones. Smoking cessation products and therapies include, but are not limited to, behavior modification therapies (e.g., counseling by a physician or other health care professional or participating in a self-help group), use of one or more nicotine replacement therapies (e.g., a nicotine transdermal patch, a nicotine chewing gum, a nicotine inhaler, a nicotine nasal spray, a nicotine sublingual tablet, or a nicotine lozenge), administration of one or more pharmaceutical products known to be useful as a smoking cessation aid (e.g., bupropion, nortriptyline, or clonidine, varenicline, cytisine, etc.), use of a cigarette substitute (e.g., a vaporizer or an electronic cigarette), and use or one or more alternative medical therapies (e.g., hypnosis, au herbal preparation such as kava or chamomile, acupuncture, or laser therapy). Alternatively, smoking cessation may be unaided (e.g., the pregnant female subject may abruptly quit smoking without assistance, commonly referred to as quitting “cold turkey,” or may gradually reduce her daily intake of nicotine without assistance),

As a particular example, one or more smoking cessation products or therapies may be used prior to and/or concurrently with the administration of 17-HPC: Specifically, the pregnant female subject may be required to attempt to stop smoking before receiving 17-HPC, and may only receive 17-HPC if her attempt at smoking cessation fails. In addition, or alternatively, the pregnant female subject may be required to use one or more smoking cessation products or therapies together with therapy with 17-HPC.

### V. Smoking-Related Diseases and Disorders

Exposure to tobacco smoke, specifically smoking by a subject, is known to contribute to various diseases or disorders. Smoking-related diseases and disorders include smoking-related cardiovascular diseases and disorders and smoking-related pulmonary diseases and disorders. For example, the smoking-related disease may be hypertension, angina, congestive heart failure (CHF), left ventricular hypertrophy (LVH), atherosclerosis, asthma, emphysema, cancer, benign tumors, chronic obstructive pulmonary disease (COPD), bronchitis, stroke, peripheral vascular disease, abdominal aortic aneurysm, decreased bone density, fracture, or sudden infant death syndrome (SIDS). Without being bound to a particular theory, it is currently believed that administration of a steroid hormone such as a progestogen (e.g., 17-HPC) to a subject known to exhibit the risk factor of smoking may reduce the risk and/or occurrence of one or more smoking-related diseases or disorders in the subject. A pharmaceutical formulation may be administered generally in accordance with the dosage regimens and pharmaceutical dosage forms detailed elsewhere herein. In accordance with such embodiments, the present invention is generally directed to a method of treating one or more smoking-related diseases or disorders which comprises administering a pharmaceutical composition comprising a progestogen to a subject suffering from a smoking-related disease.

Having described the invention in detail, it will be apparent that modifications and variations are possible without departing from the scope of the invention defined in the appended claims.

The following examples are included to demonstrate preferred embodiments of the invention. It will be appreciated by those of skill in the art that the techniques disclosed in the following examples represent techniques which function well in the practice of the invention, and thus can be considered to constitute preferred modes for its practice. However, those of skill in the art should, in light of the instant disclosure, also appreciate that many changes can be made in the specific embodiments that are disclosed, while still obtaining like or similar results, without departing from the scope of the invention. Thus, the examples are exemplary only and should not be construed to limit the invention in any way. To the extent necessary to enable and describe the instant invention, all references cited are herein incorporated by reference.

### Example 1

In a double-blind, placebo-controlled trial, 463 pregnant women having a documented history of spontaneous preterm delivery received weekly injections of 250 mg 17alpha-hydroxyprogesterone caproate (17-HPC) or an inert castor oil placebo beginning at 16 to 20 weeks of gestation and continuing until delivery or 36 weeks of gestation, whichever occurred first. The study subjects were pregnant women who had had a previous preterm delivery of a live-born singleton infant between 20 weeks and 36 weeks 6 days of gestation which was due to spontaneous preterm delivery or preterm premature rupture of the fetal membranes. A 2:1 ratio was used for the assignment of women to the 17-HPC or placebo group, since those in the placebo group would be subjected to painful injections on a weekly basis with no possibility of direct benefit. Thus, there were 310 women in the 17-HPC group and 153 women in the placebo group. The baseline characteristics of the women in the two groups were similar in terms of the mean duration of gestation of the qualifying delivery, the mean duration of gestation at the time of randomization, race or ethnic group, marital status, body mass index (BMI), educational level, smoking status, and substance use during pregnancy. The women in the placebo group had had more previous preterm deliveries (mean of 1.6 vs. 1.4 in the 17-HPC group). 91.5% of the study participants were compliant with all of their injections, noncompliance being defined as a gap of 10 days or more between any two injections, and there was no difference in the rate of compliance between the placebo group and the 17-HPC group. Preterm delivery was defined as delivery at less than 37 completed weeks (259 days) of gestation, calculated on the basis of the date of the last menstrual period and ultrasonography results.

Outcome data were available for 459 of the 463 subjects. Treatment with 17-HPC significantly reduced the risk of delivery at less than 37 weeks of gestation, with an incidence of preterm delivery of 36.3% (111/306) in the 17-HPC group as compared to 54.9% (84/153) in the placebo group (P<0.001). Treatment with 17-HPC also significantly reduced the risk of delivery at less than 35 weeks of gestation (incidence: 20.6% (63/306) in the 17-HPC group; 30.7% (47/153) in the placebo group; P=0.02), and the risk of delivery at less than 32 weeks (incidence: 11.4% (35/306) in the 17-HPC group; 19.6% (30/153) in the placebo group; P=0.02). The effectiveness of 17-HPC in this study suggested that only 5 to 6 women with a level of risk for preterm delivery similar to that of the study subjects would need to be treated in order to prevent one preterm delivery before 37 weeks gestation, and that 12 women with a similar risk level would need to be treated in order to prevent one delivery before 32 weeks of gestation. Thus, treatment with 17-HPC provided protection against both early and later preterm delivery.

The rates of several complications of prematurity were also decreased among infants of women in the 17-HPC group, and treatment with 17-HPC resulted in improved neonatal outcomes. Among the infants of women treated with 17-HPC, there was a significant reduction in the risk of a birth weight of less than 2500 g (incidence: 27.2% (82/301) in the 17-HPC group vs. 41.1% (62/151) in the placebo group; P=0.003), and a non-significant reduction in the risk of a birth weight of less than 1500 g (incidence: 8.6% (26/301) in the 17-HPC group vs. 13.9% (21/151) in the placebo group; P=0.08). In addition, infants of women treated with 17-HPC had lower rates of necrotizing enterocolitis (incidence: 0% (0/305) in the 17-HPC group vs. 2.6% (4/152) in the placebo group; P=0.01), intraventricular hemorrhage of any grade (incidence: 1.3% (4/305) in the 17-HPC group) vs. 5.2% (8/153) in the placebo group), and need for supplemental oxygen (incidence: 14.9% (45/303) in the 17-HPC vs. 23.8% (36/151) in the placebo group. However, there was no significant difference between groups in the rate of intraventricular hemorrhage of grades 3 to 4 specifically. Rates of infant death, transient tachypnea in the newborn, respiratory distress syndrome, bronchopulmonary dysplasia, need for ventilatory support, retinopathy of prematurity, and patent ductus arteriosus were slightly but not significantly lower in the progesterone group. There were 17 neonatal deaths. 16 of these were due to complications of prematurity and 1 to intrapartum hypoxia subsequent to uterine rupture.

The above-described study and results were described in Meis et al. (New England J. Medicine, 348(24):2379-2385 (2003)).

In a follow-up study, the neurodevelopmental and other health outcomes of the children of the mothers who participated in the above-described trial were systematically characterized. 278 of the surviving children were available for evaluation (194 in the 17-HPC group and 84 in the placebo group). The mean age at follow up was 48 months. No significant differences between the 17-HPC group and the placebo group were found with respect to health status or physical examination (including genital anomalies, physical abnormalities, diagnosis with various diseases and disorders, height, weight, head circumference, blood pressure, and physical growth). In addition, scores for gender-specific roles (as measured by the Preschool Activities Inventory) were within the normal range and similar between the 17-HPC and placebo groups. There were also no difference in the Ages and Stages Questionnaire, which is used to assess development in five areas (communication, gross motor, fine motor, problem solving, and personal/social), between the two groups. Thus, 17-HPC appears to be safe for the fetus when administered to the mother by weekly intramuscular injection during the second and third trimesters of pregnancy. The follow-up study is described in Northen et al. (Obstetrics & Gynecology, 110(4):865-872 (2007), which incorporated by reference herein for all relevant purposes).

### Example 2

In the clinical trial described in Example 1, 22.6% (70/310) of subjects in the 17-HPC group and 19.6% (30/153) of subjects in the placebo group smoked during pregnancy. 17-HPC significantly reduced the risk of preterm delivery among women who smoked during pregnancy, measured by the proportion of deliveries at less than 37 weeks of gestation (odds ratio (OR)=0.21). Specifically, in the placebo group, 70% (21/30) of the subjects who smoked during pregnancy delivered at less than 37 weeks of gestation, compared to 51.2% (63/123) of subjects who did not smoke during pregnancy. Thus, it was apparent in the data that smoking increased the risk for preterm delivery, measured either as delivery prior to 37 weeks of gestation. In subjects receiving 17-HPC, on the other hand, only 32.9% (23/70) of those who smoked during pregnancy delivered at less than 37 weeks of gestation, as compared to 38.3% (92/240) of those who did not smoke during pregnancy. Table 1 summarizes these results.

TABLE 1
Effects of 17-HPC on Preterm Delivery
(<37 weeks of gestational age)

| | | | Odds |
|---|---|---|---|
| | 17-HPC | Placebo | Ratio |
| | (N) | (N) | (95% CI) |
| Smoking during pregnancy | 32.9% | 70% | 0.2097 |
| | (23/70) | (21/30) | (0.0830, |
| | | | 0.5297) |
| Not smoking during | 38.3% | 51.2% | |
| pregnancy | (92/240) | (63/123) | |

Thus, 17-HPC protects against the risk of preterm delivery in women who smoke during pregnancy.

1. A method for reducing the occurrence of preterm delivery, the method comprising:
administering a pharmaceutical composition comprising a steroid hormone to a pregnant female subject having no history of preterm delivery and exhibiting one or more risk factors for preterm delivery selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

2. A method for reducing the occurrence of preterm delivery, the method comprising:
administering a pharmaceutical composition comprising a steroid hormone to a pregnant female subject having no history of preterm delivery, wherein the pregnant female subject is at risk for preterm delivery due to exposure to tobacco smoke.

3. The method of claim 2 wherein the exposure to tobacco smoke comprises smoking by the pregnant female subject during pregnancy.

4. A method for reducing the occurrence of one or more conditions selected from the group consisting of delivery of low birth weight neonates, delivery of small for gestational age neonates, pregnancy-related complications, fetal mortality, neonatal morbidity, neonatal mortality, infant morbidity, infant mortality, childhood developmental delays, and combinations thereof, the method comprising:
administering a pharmaceutical composition comprising a steroid hormone to a pregnant female subject having no history of preterm delivery and exhibiting one or more risk factors for the one or more conditions, the one or more risk factors selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

5. The method of claim 2 further comprising evaluating whether the pregnant female subject exhibits the one or more risk factors.

6. The method of claim 2 wherein the steroid hormone is a progestogen.

7. The method of claim 2 wherein the pharmaceutical composition is administered beginning at between about 1 week and about 35 weeks of gestation.

8. The method of claim 7 wherein administration continues to about 37 weeks of gestation, or delivery.

9-12. (canceled)

13. The method of claim 2 wherein the pregnant female subject is a primigravida.

14. The method of claim 2 wherein the pharmaceutical composition is administered at an interval exceeding once per week.

15. (canceled)

16. The method of claim 2 wherein the pharmaceutical composition is administered once monthly.

17. The method of claim 2 wherein the pharmaceutical composition is administered once weekly.

18. The method of claim 2 wherein the pharmaceutical composition is administered at an interval of less than one week.

19. The method of claim 2 wherein the pharmaceutical composition is administered by a method selected from the group consisting of injections, rectal delivery, transdermal delivery, intravaginal delivery, and oral delivery.

20. (canceled)

21. The method of claim 19 wherein the injection is a subcutaneous or intramuscular depot injection.

22-23. (canceled)

24. The method of claim 6 wherein the progestogen is a progestin.

25. The method of claim 6 wherein the progestogen is selected from the group consisting of a pregnane or a derivative thereof, a norpregnane or a derivative thereof, an estrane or a derivative thereof, a gonane or a derivative thereof, and combinations thereof.

26. The method of claim 6 wherein the progestogen is selected from the group consisting of progesterones, retroprogesterones, 17alpha-hydroxyprogesterone derivatives, 17alpha-hydroxynorprogesterone derivatives, 19-norprogesterone derivatives, 19-nortestosterone derivatives, and combinations thereof.

27. The method of claim 6 wherein the progestogen is selected from the group consisting of 17alpha-hydroxyprogesterone or a derivative thereof, natural progesterone, dydrogesterone or a derivative thereof, medrogestone or a derivative thereof, medroxyprogesterone or a derivative thereof, megestrol or a derivative thereof, chlormadinone or a derivative thereof, cyproterone or a derivative thereof, gestonorone or a derivative thereof, nomegestrol or a derivative thereof, demegestone or a derivative thereof, promegestone or a derivative thereof, nestorone or a derivative thereof, trimegestone or a derivative thereof, norethisterone or a derivative thereof, lynestrenol or a derivative thereof, ethynodiol or a derivative thereof, norgestrel or a derivative thereof, levonorgestrel or a derivative thereof, desogestrel or a derivative thereof, etonogestrel (3-keto-desogestrel) or a derivative thereof, gestodene or a derivative thereof, norgestimate or a derivative thereof, norelgestromin (17-deacetyl norgestimate) or a derivative thereof, dienogest or a derivative thereof, drospirenone or a derivative thereof, norethindrone or a derivative thereof, norethynodrel or a derivative thereof, and combinations thereof.

28. (canceled)

29. The method of claim 27 wherein the derivative of 17alpha-hydroxyprogesterone is a carboxylic acid ester of 17alpha-hydroxyprogesterone.

30. The method of claim 2 wherein the pharmaceutical composition comprises 17alpha-hydroxyprogesterone caproate (17-HPC).

31. The method of claim 2 wherein the pharmaceutical composition is administered to the pregnant female subject as a depot injection at an interval exceeding once weekly, wherein the composition comprises from about 100 mg to about 3000 mg of 17-HPC.

32. (canceled)

33. The method of claim 2 wherein the pharmaceutical composition is administered to the pregnant female subject as a once-weekly depot injection comprising from about 50 mg to about 1600 mg of 17-HPC.

34-36. (canceled)

37. The method of claim 2 wherein the pregnant female subject is at risk for preterm delivery due to a risk factor selected from the group consisting of exposure to tobacco smoke due to smoking by the pregnant female subject during pregnancy, exposure to tobacco smoke due to exposure to environmental tobacco smoke during pregnancy, exposure to tobacco smoke residue on environmental surfaces, and combinations thereof.

38. (canceled)

39. The method of claim 2 further comprising one or more additional treatments for reducing the occurrence of preterm delivery.

40. The method of claim 39 wherein the one or more additional treatments are selected from the group consisting of behavior modification, stress reduction or counseling, early access to care, administration or prescription of one or more pharmaceutical products, administration or prescription of one or more nutritional products, reduced physical activity, one or more surgical interventions, and combinations thereof.

41. (canceled)

42. The method of claim 3 further comprising the administration of one or more smoking cessation products and/or therapies prior to and/or concurrently with the administration of the pharmaceutical composition comprising a steroid hormone.

43-49. (canceled)

50. The method of claim 2 further comprising treating a smoking-related disease or disorder.

51. (canceled)

52. A kit comprising
a pharmaceutical composition comprising a steroid hormone and one or more pharmaceutically acceptable excipients; and
instructions for administering the pharmaceutical composition to a pregnant female subject to reduce the occurrence of preterm delivery, the pregnant female subject having no history of preterm delivery and exhibiting one or more risk factors for preterm delivery selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

53. A kit comprising
a pharmaceutical composition comprising a steroid hormone and one or more pharmaceutically acceptable excipients; and
instructions for administering the pharmaceutical composition to a pregnant female subject to reduce the occurrence of preterm delivery, the pregnant female subject having no history of preterm delivery, wherein the pregnant female subject is at risk for preterm delivery due to exposure to tobacco smoke.

54. The kit of claim 53 wherein the exposure to tobacco smoke comprises smoking by the pregnant female subject during pregnancy.

55. A kit comprising
a pharmaceutical composition comprising a steroid hormone and one or more pharmaceutically acceptable excipients; and
instructions for administering the pharmaceutical composition to a pregnant female subject to reduce the occurrence of one or more conditions selected from the group consisting of delivery of low birth weight neonates, delivery of small for gestational age neonates, pregnancy-related complications, fetal mortality, neonatal morbidity, neonatal mortality, infant morbidity, infant mortality, childhood developmental delays, and combinations thereof, the pregnant female subject having no history of preterm delivery and exhibiting one or more risk factors for the one or more conditions, the one or more risk factors selected from the group consisting of exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

56. (canceled)

57. The kit of claim 53 wherein the steroid hormone is a progestogen.

58-80. (canceled)

81. The kit of claim 53 wherein the pharmaceutical composition comprises 17alpha-hydroxyprogesterone caproate (17-HPC).

82-102. (canceled)

103. A method for treating a smoking-related disease or disorder, the method comprising:
administering a pharmaceutical composition comprising a progestogen to a subject suffering from a smoking-related disease or disorder.

104-105. (canceled)
